(19) United States
(12) Patent Application Publication
(10) Pub. No.: US 2026/0077125 A1
(43) Pub. Date: Mar. 19, 2026

(54) IV Tube Flushing Device

(71) Applicant: Boudreaux Medical Innovations, LLC, New Orleans, LA (US)

(72) Inventors: Zach Boudreaux, Metairie, LA (US); Alex Boudreaux, New Orleans, LA (US); John Boudreaux, New Orleans, LA (US); Karl Krumme, Bainbridge Island, WA (US); John Krumme, Bainbridge Island, WA (US)

(73) Assignee: Boudreaux Medical Innovations, LLC, New Orleans, LA (US)

(21) Appl. No.: 19/333,758

(22) Filed: Sep. 19, 2025

Related U.S. Application Data
(60) Provisional application No. 63/696,706, filed on Sep. 19, 2024.
(60) Provisional application No. 63/745,445, filed on Jan. 15, 2025.

(51) Int. Cl. A61M 5/168

(52) U.S. Cl. CPC A61M 5/16813; A61M 2205/3331

(57) ABSTRACT

An intravenous (IV) tube flushing device for safely delivering medication flush without risk of accidental over-administration comprises multiple embodiments of a normally-closed system that prevents uncontrolled fluid flow. One embodiment comprises a spring-loaded push-button valve that temporarily opens to allow flow only while depressed by the user. Upon release, the valve immediately returns to its closed position, eliminating the risk of forgotten open clamps. In another embodiment, the device combines traditional roller clamp functionality with an integrated flush mechanism, featuring a hinged clamp design, wherein IV tubing is secured in a channel with multiple contact points. A spring-biased roller element provides variable flow control, while a pivotable flush actuator temporarily withdraws the roller element to allow unobstructed flow for flushing operations. Both embodiments address the critical safety issue of accidental IV fluid over-administration that can occur when healthcare providers forget to close roller clamps after flushing medication from IV lines.

### CROSS-REFERENCE TO RELATED APPLICATIONS

This application claims priority to U.S. Provisional Patent Application Ser. No. 63/696,706, filed Sep. 19, 2024, and U.S. Provisional Patent Application Ser. No. 63/745,445, filed Jan. 15, 2025, the entirety of which are incorporated by reference as if fully disclosed herein.

### BACKGROUND OF THE INVENTION

Intravenous (IV) therapy is a critical medical procedure widely used in hospitals, clinics, and home care settings to deliver fluids, medications, and nutrients directly into a patient's bloodstream. IV tubes are used to conveniently deliver medications to patient by using a syringe to inject medication into an injection port. After injection into the port, the medication will often remain in the IV tubing below the injection port until flushed. The flushing process requires another fluid, typically saline, to flow behind the medication; either through the injection port or, more conveniently, by allowing the IV bag to flow. The speed of fluid administration via an IV bag is commonly controlled through a roller clamp that allows saline to flow at controllable amounts. When flushing an IV line via the IV bag, the roller clamp is opened up to temporarily allow a greater flow of saline until the remaining medication is gone.

While effective and more convenient than preparing a saline syringe to flush the system thought the injection port, the IV bag method of flushing is prone to accidental over-administration of IV fluid. Due to hectic nature of a healthcare roles, providers can forget to close the roller clamp after the system is properly flushed and allow unintended amounts of saline or other IV contents to be administered to the patient. The over-administration of IV contents, particularly saline can be harmful, and even deadly, to fluid restricted patients, especially smaller or pediatric patients. Mixing large amounts of saline with a small amount of blood in a patient's system can lead to heart failure and blood dilution causing hemodynamic and oxygen delivery issues. Worse yet, these issues can occur quickly and leave providers very little time to correct the accidental IV over-administration.

Current methods of combating accidental over administration have meaningful downsides. Using smaller bags of fluid for smaller patients and using slow drip (micro-drip) tubing provides slower IV administration than traditional IV tubing. This works increases the amount of time it takes for a critical over administration to occur. However, this strategy only mitigates the risk as an over-administration can still occur if the micro-drip tubing is left open for an extended period. Further, when micro-drip tubing is at maximum flow, it can be difficult to identify the high flow rate because the drip chamber is small and often difficult to view due to glare. The flushing method of filling an empty syringe with saline and flushing the IV line via the injection port is a safe and effective strategy but takes more time and effort than simply opening the roller clamp. In a busy healthcare environment where providers are managing multiple patients, efficient use of time is a necessity. Thus, most practitioners opt to use the roller clamp method over the injection port method. Therefore, a quick, time-efficient, fail-proof method of flushing IV lines is needed.

### DESCRIPTION OF THE PRIOR ART

The present invention has many advantages over the current state of the art. Murphy et al. (U.S. Pub. No. 2008/0091150) discloses several embodiments of IV regulators with integral flushing mechanisms. In one embodiment (FIG. 1-4), a pneumatic plunger is used to disengage the roller clamp for a timed flush. In another embodiment (FIG. 10-13), collapsible bladders are alternately filled and emptied to provide a set volume of flush fluid. Of particular relevance is the embodiment of FIG. 5-9, which employs a pivoting lever, torsion spring, and rotary damper to temporarily disengage the roller clamp and allow a timed bolus flush before returning to a regulated flow position.

While Murphy et al. represents an advance over manual roller-clamp operation, the FIG. 5-9 embodiment suffers from several deficiencies. The device provides only a single point of contact between the roller clamp and the IV tubing. This arrangement does not securely hold the tubing in position, allowing the tubing to shift or slide off the roller clamp during use. This instability risks uncontrolled infusion. The surface opposite the roller clamp is limited and non-uniform. As a result, the tubing is not evenly compressed, and consistent occlusion of the lumen may not be achieved. This may lead to inconsistent flow control. The lever and rotary damper assembly significantly increases the size of the device. The Murphy device relies on the rotary damper to gradually return the lever to a closed position. This prevents immediate shutoff of flow when the actuator is released, raising safety concerns where rapid cessation of fluid delivery may be required.

The most concerning flaw of the Murphy device is its large footprint, which includes multiple protruding components that can easily catch on other equipment or lines in a crowded hospital environment. These protruding handles pose a particular hazard by becoming entangled with IV tubes or electrical wires, requiring healthcare providers to intervene, potentially delaying the delivery of other critical services. More alarmingly, such snags and tangles may cause direct harm to patients. Consequently, the Murphy device presents unacceptable risks in hospital and clinical settings.

Accordingly, although Murphy et al. acknowledges the need for integrated flushing mechanisms, its designs fail to adequately secure the tubing, do not provide a consistent contact surface for reliable occlusion, occupy an unnecessarily large footprint, and lack the ability to immediately shut off upon release. These deficiencies highlight the need for improved IV regulators that overcome these shortcomings.

US Patent Publication No. US2010/217232 A1 (“the '232 device”) discloses a bypass valve for use in an IV system. In the present invention, the flush bypass line is activated by a spring-loaded button, clip, pin or other normally-closed component. When activated, fluid is released and flows through the line by gravity, rather than being pressure-driven. In contrast, the '232 device requires a pressure gradient (created either by squeezing the IV bag upstream or by applying negative pressure downstream with a syringe) to open an internal pressure-activated valve. The present invention can operate even when the IV bag contains only a low fluid volume and does not require the additional equipment needed by the '232 device. Furthermore, the '232 device relies on internal mechanisms that may be difficult to repair and may introduce small parts into the IV line if the device fails. By contrast, the present invention uses an external valve that can be easily replaced and does not introduce extra components into the internal tubing of the system.

In the '232 device, generating a pressure gradient is difficult when the IV bag contains only a small volume of fluid. Because the bag is collapsible, positive pressure cannot be created with low volume. Negative pressure activation, on the other hand, requires the use of a syringe or other equipment. The present invention eliminates both problems: it permits flow with a low fluid volume in the bag and does not require auxiliary equipment. The '232 device also requires the user either to stand and squeeze the IV bag—typically hanging about six feet above the ground—or to connect a syringe and perform multiple steps to flush the system. The present invention avoids these inconveniences by locating the safety valve at a lower and more accessible position for medication administration. In addition, the valve's position can be adjusted by a tubing clip attached to the roller clamp.

In the present invention, priming of the two parallel lines occurs with substantially equal flow, reducing the amount of air trapped in the system. When open, the safety valve provides full and even flow through the bypass line, comparable to the roller clamp line. By contrast, in the '232 device, depressing the ball valve creates turbulent flow, causing less fluid to enter the bypass flush line relative to the roller clamp line. This uneven flow can result in air entrapment in the bypass line, requiring additional flushing of the entire line to remove the air.

The bypass button-activated valve of the present invention requires no internal modification of the IV tubing. Standard tubing can be used, with the spring-loaded valve attached externally. The '232 device, in contrast, requires a valve system built into the tubing itself. This internal placement increases the risk of malfunction and introduces a safety hazard if internal parts—such as springs or balls—were to detach and enter the IV line, potentially reaching the patient.

The external valve of the present invention also allows the practitioner to move the system in both directions to visually confirm the integrity of the IV line. The '232 device, however, incorporates opaque components into the tubing that can obstruct visual inspection. Additionally, the internal valve of the '232 device is fixed in place, preventing the user from repositioning it to inspect the tubing.

The present invention provides relatively consistent fluid flow through the IV bypass line, since the same gravity-driven conditions are present each time the button is depressed. By contrast, the '232 device requires creation of a pressure gradient by squeezing the IV bag, which produces variable pressures depending on the force applied and the remaining fluid volume. This inconsistency makes it nearly impossible to accurately estimate pressure or flow, as the drip chamber often becomes a continuous stream, eliminating the ability to count drops to approximate volume. While the '232 device can also use a syringe to create negative pressure, this poses risks when fluid levels are low: the syringe can draw in large volumes of air, which is dangerous when introduced into an IV line. By comparison, the button-activated system of the present invention poses far less risk of air entrapment, as it relies only on gravity rather than mechanically generated negative pressure.

### SUMMARY OF THE INVENTION

In accordance with some embodiments, the present invention is a human error proof IV flush system comprising a momentary or push-button valve incorporated into an IV line.

### BRIEF DESCRIPTION OF DRAWINGS

The foregoing and other objects, features, and advantages of the invention are apparent from the following detailed description taken in conjunction with the accompanying drawings in which like parts are given like reference numerals and, wherein:

FIG. 1A depicts an IV tubing system in accordance with embodiments of the invention.

FIG. 1B depicts an IV tubing system incorporating an improved IV flushing system in a parallel line in accordance with embodiments of the invention.

FIG. 2 depicts a push-button valve of an improved IV flushing system in accordance with embodiments of the invention.

FIG. 3 depicts an exploded view of a push-button valve of an improved IV flushing system in accordance with embodiments of the invention.

FIG. 4 depicts a cross-sectional view of a push-button valve flush device with an IV tube in an open configuration in accordance with embodiments of the invention.

FIG. 5 depicts a cross-sectional view of a push-button valve flush device with an IV tube in a closed configuration in accordance with embodiments of the invention.

FIG. 6 depicts an angled view of an improved IV flush device in accordance with embodiments of the invention.

FIG. 7 depicts an angled view of a lower portion of an improved IV flush device in accordance with embodiments of the invention.

FIG. 8 depicts a cross-sectional side view of an improved IV flush device in a closed position accordance with embodiments of the invention.

FIG. 9 depicts a cross-sectional side view of an improved IV flush device in an open position with IV tubing placed in the device in accordance with embodiments of the invention.

FIG. 10 depicts a cross-sectional side view of an improved IV flush device with IV tubing placed in the device in a closed position in accordance with embodiments of the invention.

The images in the drawings are simplified for illustrative purposes and are not depicted to scale. Within the descriptions of the figures, similar elements are provided similar names and reference numerals as those of the previous figure(s). The specific numerals assigned to the elements are provided solely to aid in the description and are not meant to imply any limitations (structural or functional) on the invention.

The appended drawings illustrate exemplary configurations of the invention and, as such, should not be considered as limiting the scope of the invention that may admit to other equally effective configurations. It is contemplated that features of one configuration may be beneficially incorporated in other configurations without further recitation.

### DETAILED DESCRIPTION OF THE INVENTION

The embodiments of the disclosure will be best understood by reference to the Figures, wherein like parts are designated by like numerals throughout. It will be readily understood that the components, as generally described and illustrated in the Figures herein, could be arranged and designed in a wide variety of different configurations or be entirely separate. Thus, the following more detailed description of the embodiments of the device of the disclosure, as represented in the Figures is not intended to limit the scope of the disclosure, as claimed, but is merely representative of possible embodiments of the disclosure

The following description sets forth numerous embodiments and parameters. It should be recognized, however, that such description is not intended as a limitation on the scope of the present invention but is instead provided as a description of exemplary embodiments. Various modifications to the examples described will be readily apparent to those of ordinary skill in the art, and the general principles defined may be applied to other examples and applications without departing from the spirit and scope of the invention. Thus, the present invention is not intended to be limited to the examples described herein but is to be accorded a scope consistent with the claim

The invention is generally directed to a system for safely flushing the line of an Intravenous (IV) therapy after a medication has been injected into the line via an injection port or other entry means. The system may also allow for the bypass of the traditional IV administration control means for other purposes.

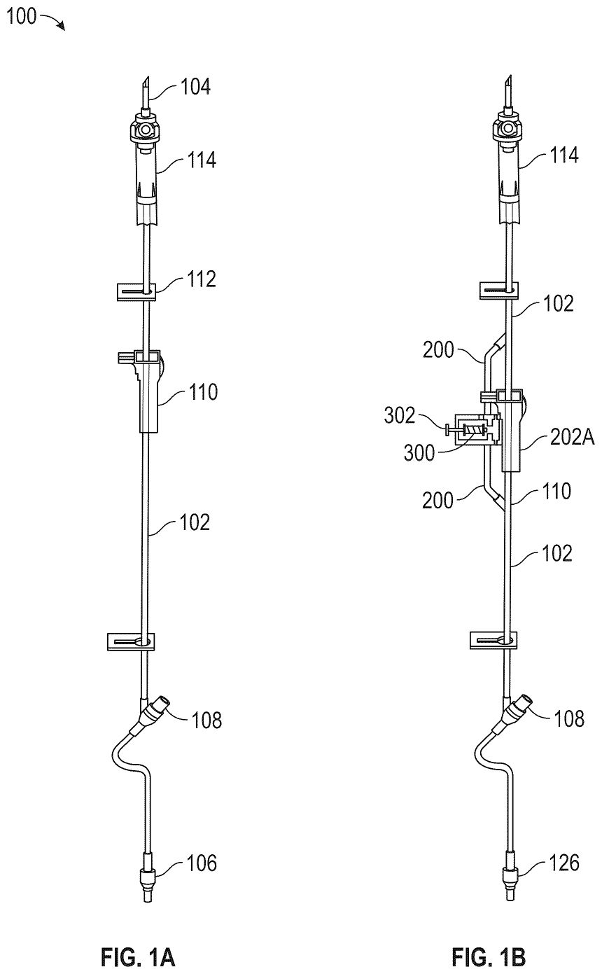

FIG. 1 depicts a traditional IV therapy system comprising a single, main line IV system. The common IV system includes a main line 102 that extends from an IV bag spike 104 to a dispensing means such a needle or Luer connecter 106. The Luer connector 106 is a connection mechanism that can be quickly and removably attached to a compatible needle. The common IV system may also include an injection port 108, a flow regulator 110, a slide clamp 112, a drip chamber 114 or a solution filter 116. The injection port 108 allows for the introduction of medicine into the main line 102 of the IV system for eventual delivery to a patient. A drip chamber 114 is a sealed, inline chamber that is used to allow gas to separate from the IV fluid before the fluid travels downstream, in order to prevent embolism. The drip chamber 114 also allows for the visualization of the flow rate of the IV fluid administration. Specifically, the number of drops per minute in the drip chamber can be used to calculate the flow rate of the IV fluid administration.

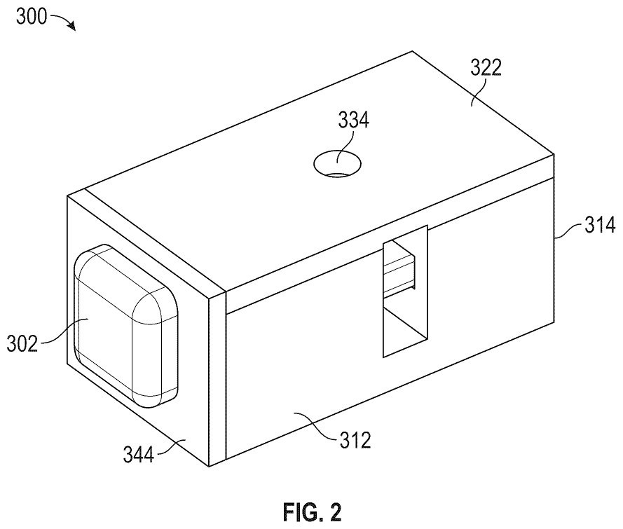

The flow regulator 110 provides a means of starting, stopping and precisely controlling the amount of fluid flow through the main line 102. Common flow regulators include roller clamps, slide clamps, pinch clamps or microflow regulators. FIGS. 1 and 2 depict an IV therapy system utilizing a roller clamp 110 as a flow regulation means.

IV therapy systems are used for several purposes in the medical field, one of the most common being the rapid administration of medication to a patient via the bloodstream called an IV Push. IV therapy systems commonly include injection ports, such as those displayed in FIGS. 1 and 2, that allow medications to be introduced into the IV mainline 102 with a syringe. The medication travels through the mainline and into the bloodstream of a patient. After an IV push is completed, it is common for small amounts of medication to remain in the IV mainline. Remnant medication is undesirable because it means a portion of the prescribed dose did not reach the patient. Further, the remnant medication may become unblocked at a later time and be introduced to the patient at an unwanted time. To prevent these situations, providers will flush the IV line with an amount of additional IV fluid to flush the remnant medication out of the mainline. The most common method of flushing an IV line is to increase the flowrate of the IV by using the system's flow regulations means). For example, the roller clamp of an IV system may be opened to increase the flow of the IV fluid and push the downstream remnant medication out of the line. After the remnant medication is removed from the line, a provider returns the flowrate pre-flush process amount via the same flow regulation means.

While effective and convenient, flushing the line using the flow regulation means and IV bag fluid is prone to accidental over-administration of IV fluid caused by a provider forgetting to close or reduce the IV flow after medication flush is completed. In the hectic environment of hospital and clinics, this a not an uncommon occurrence. The present invention provides a safer way to flush medication using the IV bag method

One embodiment of the present invention comprises a bypass line attached to the mainline of an IV system at points on either side of the flow regulations means. The bypass line includes a mechanical or electromechanically normally closed valve that allows for temporary flow of the IV fluid at a high flowrate only will a provider interacts with the actuation means of the valve. In some embodiments, the invention comprises a mechanical or electromechanically normally closed valve incorporated in the mainline of the IV system without use of a bypass line.

FIG. 2 depicts an embodiment of the invention incorporated into an IV system. In the depicted embodiment, the device includes a bypass line 200 that is connected to IV system mainline 102. The bypass line 200 may be connected to mainline 102 at points on either side of the flow regulation means 110. The bypass line 200 operates to allow a flow of IV fluid around the flow regulation means 110. The internal diameter of the bypass line can be selected based on the desired flowrate of the bypass line. For example, the internal diameter of the bypass line may be selected to create a flow rate desirable for flushing medication in the downstream mainline. The internal diameter of the bypass line may selected to match the internal diameter of the mainline.

The pre-flow regulator connection point 202a and the post-flow regulator connection point 202b of bypass line 200 may be any point along the mainline that allows the bypass line 200 to travel around the flow regulation means 110. See FIG. 2.

As previously discussed, the IV flushing device may include a mechanical or electromechanically normally closed safety valve that allows for temporary flow of the IV fluid at a high or maximum flowrate. The normally closed valve may be a momentary valve, push-button valve, solenoid valve or any other normally closed valve with a temporary open functionality. The safety valve may be placed anywhere along bypass line 200. In some embodiments, the safety valve is movable along the bypass line such that it may be adjusted on the fly to suit a provider's height or position. The safety valve is configured such that its default state is to block the flow of fluid through the IV line. The safety valve is further configured to allow flow through the bypass line when a user actuates an actuator. Upon release or non-actuation of the actuator by a user, the safety valve is configured to return to its default state and block the flow of fluid through the bypass line. The safety flushing valve may have a temporary active “flow” or “flush” or “on” state that allows for a flow of IV fluid sufficient to flush the downstream mainline. The default state of the safety flushing valve is “closed” or “off” state wherein flow through or across the valve is prevented.

The combination of a normally closed/temporary open safety valve in a bypass lined setup to bypass the traditional roller clamp provides a safe and fail proof system for quickly allowing a higher flow of IV fluid to clean a downstream portion of an IV line. The provider simply activates the safety valve for a brief period via a positive input to allow more fluid to flow from the IV line and the then deactivates the safety valve and IV flow by releasing or ceasing the positive input on the safety valve. The normally closed configuration of the safety valve prevents unintentionally leaving an IV flow at an unwanted or dangerous flow rate.

As depicted in FIG. 2, the safety valve may comprise a push-button valve 300 located anywhere along the bypass line 200. The default state of the push-button valve 300 is to clamp the bypass line from the out of the line to prevent the flow of IV fluid through the bypass line 200. When button 302 is pressed the, the bypass line is unblocked, and IV fluid can flow through the bypass line 200 at a rate necessary to flush the mainstream line below connection point 202b.

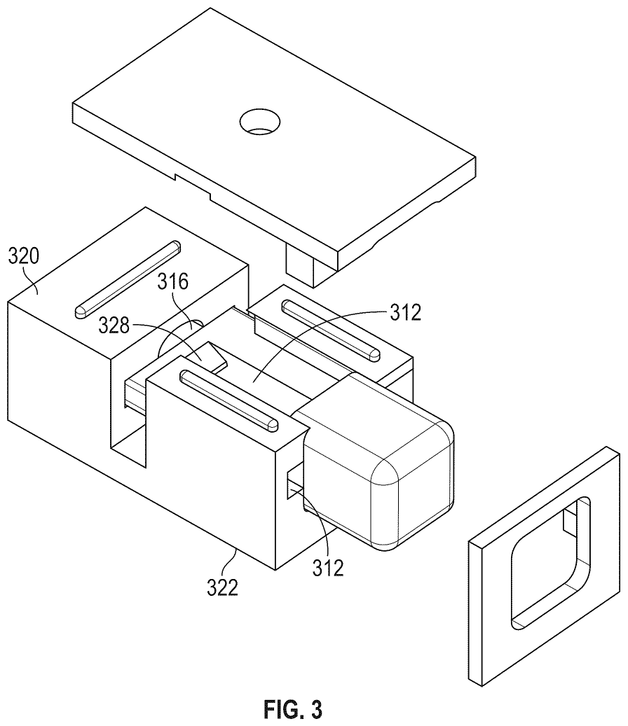

As shown in FIGS. 3-5, push-button valve 300 comprises a button 302 attached to a paddle 304, a spring 306, a main housing 308, and top portion 310.

As shown in FIGS. 3-5, main housing 308 may be generally rectangular in configuration and may comprise button side 313 and a spring side 314. Main housing 308 may further a paddle channel 312 extending internally from the button side 313 to the spring side 314 of main housing 308. The paddle channel 312 is configured to slidably receive the paddle 304 that is connected to button 302. A spring side 314 portion of the main housing 308 may comprise a spring channel 316 configured to receive spring 306. Spring channel 316 and paddle channel 312 overlap and extend along the same path such that spring 306 naturally pushes paddle 304 (and button 302) away from the spring side 314 of the main housing 308 through the paddle channel 312. Conversely, the pressing of button 302 forces paddle 304 to compress spring 306 within spring channel 316 towards the spring side 314 of main housing 308.

Main housing 308 may further comprises a cross channel 318 that extends through a portion of the main housing 308 starting from the top surface 320 of main housing 308 towards the bottom surface 322 of main housing 308. The cross channel 318 may be rectangular and extends in a direction that is perpendicular to paddle channel 312. The cross channel 318 divides the paddle channel 312 into a spring side portion of the paddle channel that includes the overlapping spring channel 316 and a button side portion of the paddle channel that does not include the spring channel 316. The bottom of cross channel 318 comprises a main body tubing aperture 342 that extends through the bottom surface 322 of main housing 308 and is configured to receive an IV tube.

Main housing 308 may further comprise a button channel 324 that extends from the button side 313 of main housing 308 to the cross channel 318. The button channel 324 extends along and overlaps with the button side of paddle channel 312. Button channel 324 is configured to slidably receive button 302. A face plate 344 is configured to attach to the button side 313 surface of main housing 308. Face plate 344 can help prevent over-translation of button 302 and paddle 304 out of their respective channels.

As shown in FIG. 3-5, paddle 304 and button 302 may be attached. Paddle 304 comprises a paddle tubing channel 326 through the center of paddle 304. Paddle tubing channel 326 is sized and configured to receive and IV tube running in a direction that generally perpendicular to the major face of paddle 304. Paddle tubing channel 326 is also sized and configured to allow post 330 of top portion 310 to pass through it. Paddle tubing channel 326 further comprises a paddle crimp 328 shaped to contact and pinch, crimp or compress an IV tube when the paddle is pressed toward the IV tubing.

Main housing 308 further comprises a post détente 340 extends from the button side of paddle channel 312 towards a bottom surface 316 of main housing 308. The post détente 340 may be rectangular in shape. The post détente 340 is configured to receive a portion of post 330 of top portion 310.

Top portion 310 comprises a lid 332, lid tubing aperture 334 and a post 330. Post 330 comprises a post crimp 336 shaped to contact and pinch, crimp or compress an IV tube when an IV is squeezed or compressed between post crimp 336 and paddle crimp 328. Post 330 is sized and configured to extend through the tubing channel 326 of paddle 304 and into post détente 340.

The pressing of button 302 (and therefore actuation of spring 306), puts push button valve 30 into its “flow” or “flush” or “on” state. The release of button 302 (and return of spring 306 to its natural state), puts push button valve 300 into its “closed” or “off” or “default” state.

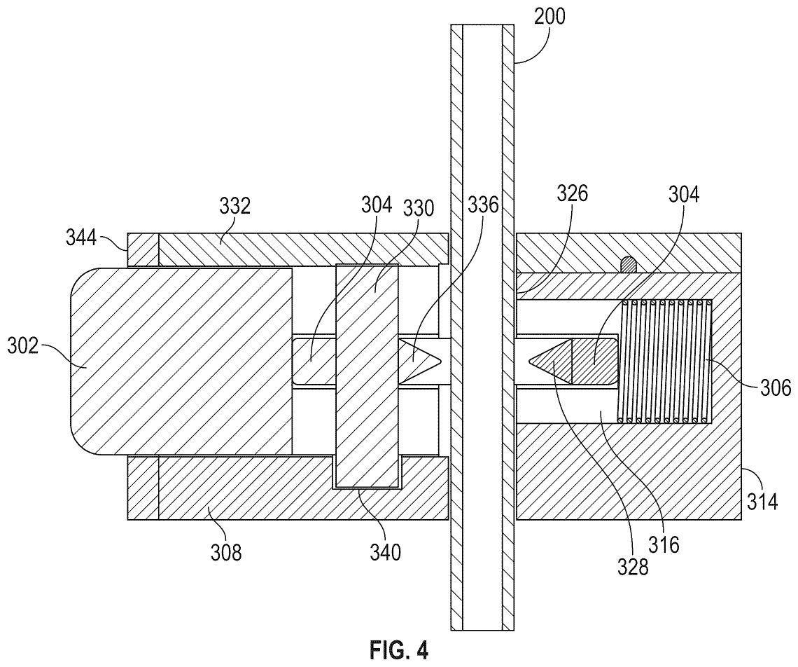

As shown in FIG. 4, when push button valve 300 is fully constructed and button 302 is pressed the paddle 304 is pushed toward the spring side of main housing 308. The causes portion of the tubing channel 326 of paddle 304 to align with lid tubing aperture 334 and main body tubing aperture 342 to define a pathway such that an IV tube can extend entirely through push button valve 300. In this “on” configuration, the space provided by the portion of the tubing channel 326 contains the paddle crimp 328 on one side and the post crimp 336 of post 330 on the other. When an IV tube is inserted into this space, it sits between the paddle crimp 328 the post crimp 336.

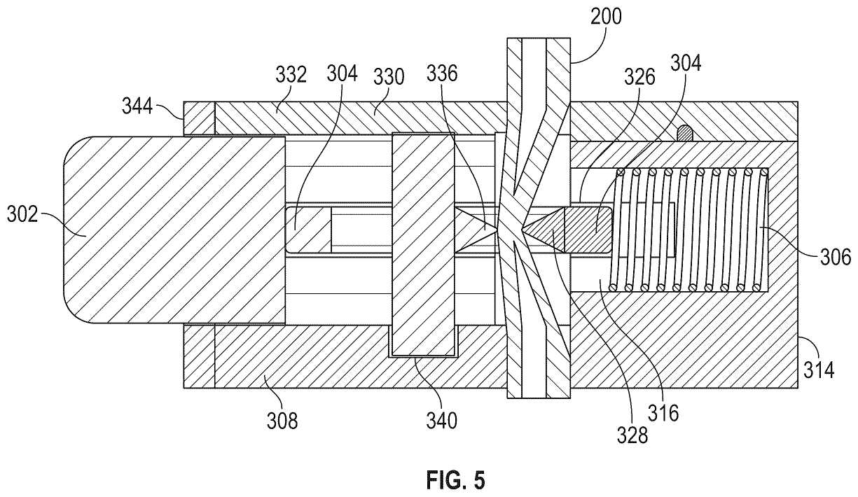

As shown in FIG. 5, when button 302 is released, and the push button valve enters an “off” configuration, spring 306 forces paddle 304 back toward the button side of the device, thereby pinching the IV tube between paddle crimp 328 the post crimp 336. This pinching action compresses the IV tube and prevents the flow of fluid through the IV tube.

In some embodiments of the invention, safety flush valve may be used on the main line of an IV system and on a bypass line of a system. In some embodiments of the invention, a safety flush valve may be used on the main line of system and no bypass line is used on the system. In some embodiments of the invention, a safety flush valve may be used on only the bypass line of the system and the main line does not include a safety flush valve.

The invention further contemplates an embodiment combining a safe and convenient IV flushing function and the function of the roller clamp.

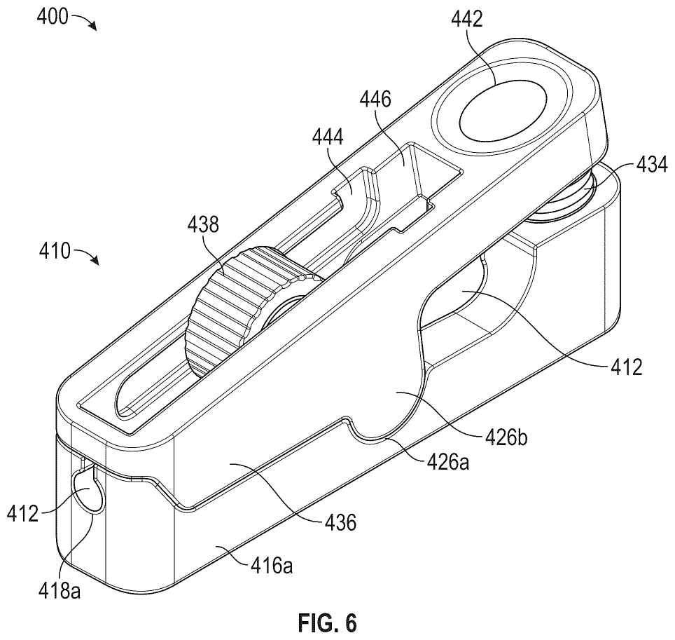

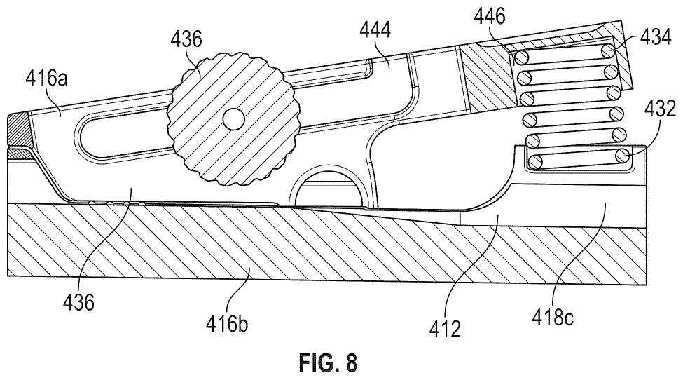

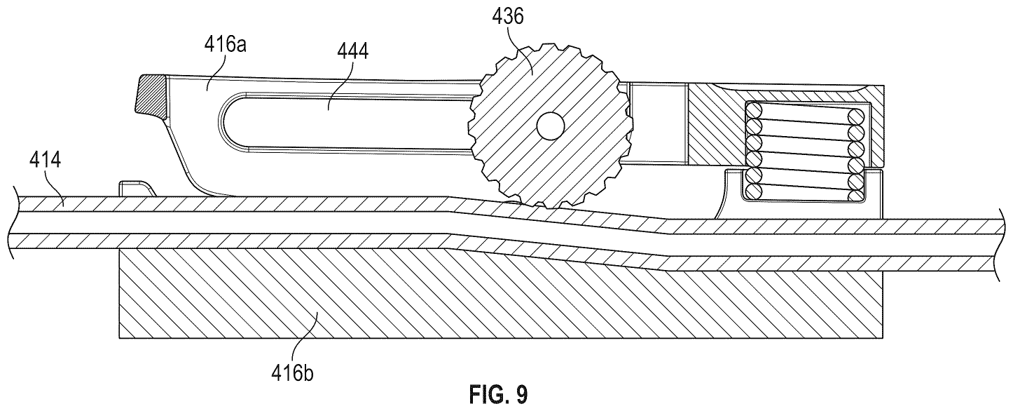

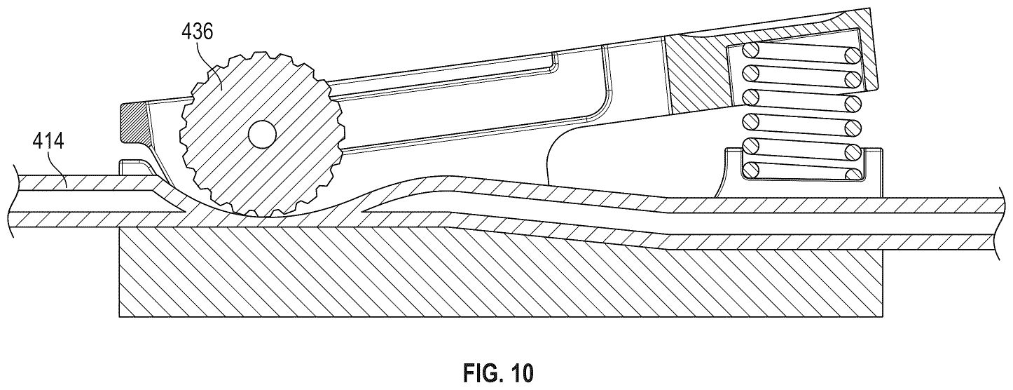

As shown in the embodiment depicted in FIG. 6-10, the device 400 comprises a main body 410 defining a channel 412 for receiving an intravenous tubing 414. The main body comprises a lower body portion 416a and an upper body portion 416b.

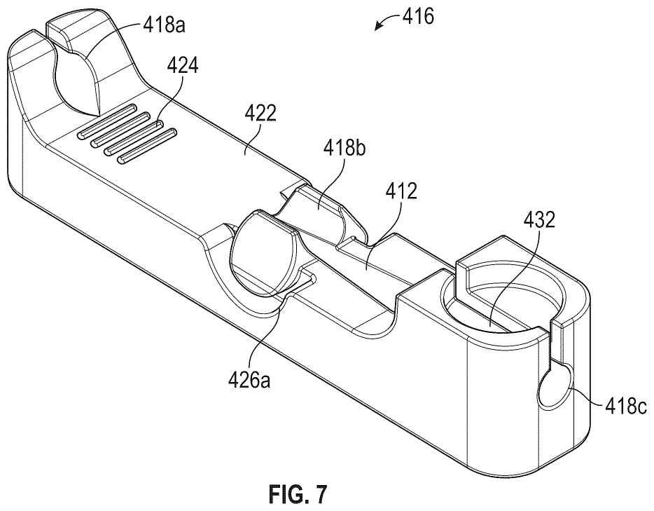

As shown in FIG. 7, in some embodiments, channel 412 may be formed in a lower body portion 416a of the main body 410 and may be shaped and dimensioned to receive and support intravenous tubing 414. For example, the channel may comprises portions that comprise a rounded groove and may comprises other portions that define semi-circular, partially circular, nearly fully circular or circular sections. As shown in the embodiment depicted in FIG. 7, the channel 412 may comprise one or more channel support sections 418 formed in lower body portion 416a. The device may comprise a forward channel support section 418a, a middle channel support section 418b and rear channel support section 418a. The channel support sections 418 engage the IV tubing 414 at multiple points along the length of the device, thereby creating correct alignment and retention of the tubing to ensure that a roller element 430 correctly contacts the IV tubing 414 before and after using the device to perform a flush. The channel support section also prevents the tubing from slipping or shifting laterally during use of the device.

As shown in FIG. 7, in some embodiments, one or more support sections may comprise a nearly full circular shape to almost completely enclose the IV tubing as to maximize security of the tubing. The sections may include a slit or break 420 in the structure to allow IV tubing 414 to be inserted or removed. This function allows the device to be installed and removed on IV tubing 414 at will and allows the device to be compatible with existing IV systems and tubing.

As shown in FIG. 7, in some embodiments, lower body portion 416a further includes an IV tubing contact surface 422, which may comprise a flat surface including an anti-slip feature such as strips 424. These features further ensure that the roller element 430 consistently contacts the IV tubing 414 to create consistent occlusion and reliable flow control across the full lumen of the tube.

As shown in FIG. 7, in some embodiments, lower body portion 416a comprises a lower hinge feature 426a that is configured to mate with a corresponding upper hinge feature 426b of upper body portion 416b of the main body 410 to form hinge 438. The lower body portion 416a further comprises a lower spring well 432 for holding a lower end of coil spring 434.

As shown in FIG. 6, 8-10, in some embodiments, bottom of upper body portion 416b comprises an angled ramp surface 436 that is configured to meet with the face of the tubing contact surface 422 of the lower portion 416a when the device 400 is closed and there is no IV tubing 414 in the device. A slot 440 exists through angled ramp surface 436 containing roller clamp element 438. The roller clamp element 438 may be generally cylindrical and freely rotatable, and operates by compressing the flexible IV tubing 414 against the rigid flat contact surface 422 disposed on the lower body portion 416a opposite the angled ramp surface 436 to variably occlude the tubing lumen and control fluid flow rate. The roller clamp element 438 is translatable along slot 440 and movement of the roller clamp element 438 along the slot allows for precise flow adjustment by rolling the cylindrical element to different positions, where complete closure stops flow entirely and partial compression permits controlled flow rates proportional to the degree of tubing occlusion. A user may incrementally reposition the roller clamp element 438 along the angled ramp surface 436 using a thumb or finger to adjust the degree of IV tubing compression and establishing a desired drip rate.

The roller clamp element 438 extends through the top of slot 440 so that a user can access roller clamp element 438 to adjust flowrate through IV tubing and extends through the bottom of slot 440 in order to contact the IV tubing. Roller clamp element 438 comprises two shafts 442 that are retained in tracks 444 formed in the upper body portion 416b and allow for the roller clamp element 438 to be rotated and translated along upper body portion 416b.

Roller clamp element 438 is biased toward the IV tubing 414 by a coil spring 434. The coil spring 434 urges the roller clamp element 438 into a default closed position in which the IV tubing 414 is fully or partially occluded. The coil spring 434 ensures that the device will immediately return to a safe, closed state wherein the IV tubing returns the flowrate previously selected by a user.

As shown in FIG. 6 and 7-10, in some embodiments, upper body portion 416b may further comprise a pivotable flush actuator 442 which is used to compress coil spring 434 and thereby manipulate roller clamp element 438. When depressed, the pivotable flush actuator 442 temporarily withdraws the roller clamp element 438 away from the IV tubing 414, allowing unobstructed fluid flow through the tubing 414 for flushing operations. Upon release, spring 434 immediately restores the roller clamp element 438 into its prior position, re-establishing the pre-set drip rate or, if the roller is at the closed end of the ramp, returning to full occlusion. This arrangement allows clinicians to flush without losing the prior flow setting, while ensuring that fluid delivery stops as soon as the flush actuator 442 is released.

As shown in FIG. 6-10, coil spring 434 is seated in a lower spring well 432 in lower body portion 416a and an upper spring well 446 in upper body portion 428. The inventions contemplates the use of other forms of spring mechanisms such as compression springs, conical compression springs, barrel springs, hourglass springs, wave springs, Belleville washers (disc springs), variable pitch compression springs, and nested (telescoping) compression springs.

As illustrated in the embodiment shown in FIG. 6-10, the main body 410 is compact, featuring smooth outer surfaces and minimal protrusions. This streamlined design reduces the device's footprint and lowers the risk of snagging on clothing, bedding, or nearby medical equipment in a crowded hospital environment. By being tangle-resistant, the device eliminates the need for healthcare providers to spend time untangling it from surrounding tubes or wires and, more importantly, helps prevent injuries that could result from pulling on lines or tubes attached to a patient. Portions of main body 410, such as pivotable actuator 442, may further include a textured or contoured surface to improve grip during use. In certain embodiments, the device 400 is configured as an after-market accessory that can be placed onto existing IV tubing 414. In other embodiments, the device 400 may be pre-assembled with the IV tubing 414, providing an integrated unit ready for clinical use. Regardless of configuration, the device 400 can be selectively repositioned along the IV line by sliding the main body 410 up or down the tubing 414 to suit the user's preferred placement. This construction provides numerous advantages over prior devices. The three-point tubing retention ensures stability and prevents slippage, while the flat contact surface 422 opposite the roller clamp element 438 enables reliable and consistent occlusion of the IV tubing 414. The spring-biased roller clamp element 438 ensures immediate shutoff when the actuator 442 is released, thereby reducing safety risks associated with uncontrolled infusion.

The embodiment depicted in FIG. 6-10, may be used in either a traditional IV therapy system comprising a single, main line IV system or an IV system comprising a bypass line attached to the mainline of an IV system at points on either side of the flow regulations means.

For the purposes of promoting an understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical”. Numerous modifications and adaptations will be readily apparent to those skilled in this art without departing from the spirit and scope of the present invention.

What is claimed is:

1. A intravenous (IV) line flow control and safety device comprising:
A main body defining a channel wherein the channel is configured to receive the tubing of an IV line and wherein the main body further comprises:
An upper body portion including a roller element that is configured to translate along the upper body portion and contact tubing of an IV line to occlude the tubing and restrict flow;
A lower body portion, that is hingedly connected to the upper body portion;
A spring element located between the upper body portion and the lower body portion, wherein the spring is configured to bias the roller element of the upper body portion towards the lower body portion;
an actuator configured to disengage the roller element away from the lower body portion.

2. The device of claim 1, wherein the channel, is configured to contact the tubing of an IV line at two or more points when the tubing of an IV line is placed in the channel.

3. The device of claim 1, wherein the channel comprises at least two channel support sections configured to retain the IV tubing when IV tubing is placed in the device.

4. The device of claim 1, wherein the channel is defined by the lower body portion and wherein the channel runs the entire length of the lower body portion.

5. The device of claim 1, wherein the actuation of the actuator is configured to be operated by two fingers.

6. The device of claim 1, the lower half comprises a flat contact surface.

7. The device of claim 6, wherein the flat contact surface comprises anti-slip features configured to prevent movement of IV tubing during operation.

8. The device of claim 3, wherein at least one of the at least two channel support section a slit configured to allow insertion and removal of IV tubing.

9. The device of claim 3, wherein at least one of the channel support sections comprises at least a semicircular cross-section

10. The device of claim 3, wherein at least one of the at least two channel support sections comprises a generally circular cross-section.

11. The device of claim 10, wherein the channel is sized and shaped to be retain the IV tubing.

12. The device of claim 1, wherein the spring element comprises a coil spring

13. The device of claim 12, wherein the coil spring is seated in a lower spring well in the lower body portion and an upper spring well in the upper body portion.

14. The device of claim 1, wherein the actuator comprises a pivotable actuator with a textured surface to improve grip during use.

15. The device of claim 1, wherein the actuator is comprised of an arm that extends less than 4 inches from the hinge such that the device has a compact configuration.

16. A safety device for safely controlling fluid flow through an intravenous (IV) line comprising:
A main body defining a channel, wherein the channel is configured to receive IV line tubing;
wherein the channel comprises at least two separate contact structures configured to retain the IV tubing when IV tubing is placed in the device;
a roller clamp element positioned configured to compress IV line tubing variably occlude a tubing lumen and control fluid flow rate.

17. A method of safely flushing an IV line comprising:
positioning an IV flow control device on IV tubing, wherein the device comprises a translatable spring-biased roller element in a default closed position, wherein the default closed position is configured to allow the roller element to wholly or partially occlude the IV tubing;
depressing an actuator to temporarily withdraw the roller element from the IV tubing against a spring bias to put the device in an open position;
allowing fluid to flow through the IV tubing in an open position; and
releasing the actuator to allow the spring bias to immediately return the roller element to its default closed position.

18. The method of claim 17, wherein the default closed position comprises a pre-set drip rate established by translating the roller element along an angled ramp surface before initiating the flush operation.
